(19) United States
(12) Patent Application Publication
(10) Pub. No.: US 2004/0028684 A1
(43) Pub. Date: Feb. 12, 2004

(54) Cancer diagnosis and assays for screening anti-cancer agents

(76) Inventors: Matthias Georg Christian Gstaiger, Basel (CH); Wilhelm Krek, Riehen (CH)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/276,016

(22) PCT Filed: May 11, 2001
(86) PCT No.: PCT/EP01/05411

(30) Foreign Application Priority Data
May 12, 2000 (GB) 0011439.7

(51) Int. Cl. A61K 39/395; C07K 14/705; C07K 16/30

(52) U.S. Cl. 424/155.1; 530/350; 530/388.8

(57) ABSTRACT

A novel RMP homologue is provided, as well as nucleic acids encoding the protein and antisense nucleic acids, Methods of diagnosing conditions dependent on RMP are provided as well as screening methods for identify agents active against conditions dependent on RMP, such as conditions dependent on amino acid regulation, cancer, neurodegeneration, muscle degeneration, immune disorders, and AIDS.

The invention relates to the field of cell proliferation, with applications in cancer diagnosis and therapy. The invention also relates to the screening of compounds for potential anti-cancer activity, whether prophylactic or therapeutic. The screening assays concerned are those which seek to mimic a part of the biochemical machinery of intact cells in vivo involved in processes of cell division, gene expression and transformation.

In the more affluent countries of the world cancer is the cause of death of roughly one person in five. The American Cancer Society in 1993 reported that the five most common cancers are those of the lung, stomach, breast, colon/rectum and the uterine cervix. Cancer is not fatal in every case and only about half the number of people who develop cancer die of it. The problem facing cancer patients and their physicians is that seeking to cure cancer is like trying to get rid of weeds. Although cancer cells can be removed surgically or destroyed with toxic compounds or with radiation, it is very hard to eliminate all of the cancerous cells. A general goal is to find better ways of selectively killing cancer cells whilst leaving normal cells of the body unaffected. Part of that effort involves identifying new anti-cancer agents.

Cancer cells have lost the normal control of the cell cycle and so divide out of control compared to normal cells. The sub-cellular machinery which controls the cell cycle is a complex biochemical device made up of a set of interacting proteins that induce and co-ordinate the essential processes of duplication and division of the contents of a cell. In the normal cell cycle, the control system is regulated such that it can stop at specific points in the cycle. The stopping points allow for systems of feedback control from the processes of duplication or division. They also provide points for regulation by environmental signals.

Gene expression plays an integral part in cell division and its control. Loss of control of cell division may in certain instances have its origin in an alteration in gene expression. Analysis of genetic alterations in cancer cells has revealed many genes which encode proteins involved in the control of cell division in some way.

Oncogenes are one family of such genes. Oncogenes are either expressed in cancer cells in a mutated form or they are over-expressed. The products of such oncogenes promote cell proliferation. The non-mutated or normally expressed version of an oncogene is known as a proto-oncogene and this is expressed in normal cells and encodes a constituent protein of the normal cellular machinery.

Another kind of gene product connected with cancer is that expressed by tumour-suppressor genes and the gene products serve to restrain cell proliferation. Mutation of a tumour-suppressor gene or loss of function of the gene product results in a loss of the normal control on proliferation and the cell divides out of control.

The study of cancer cells and their oncogenes or tumour-suppressor genes has helped to show how growth factors regulate cell proliferation in normal cells through a complex network of intracellular signalling cascades. These cascades ultimately regulate gene transcription and the assembly and activation of the cell cycle control system. As knowledge increases about the component parts of the cell cycle control machinery and how it operates, the possibilities for correcting the loss of control in cancer cells are increased. Essential points of control and essential proteins can be identified in the control hierarchy and potentially targetted with drugs to act as promoters or inhibitors, as required.

The cell cycle control system is based on two main families of proteins. The first is the family of cyclin-dependent protein kinases (CDK) of which there are a number of varieties, e.g. CDK 1 and CDK 2. CDK phosphorylates selected proteins at serine and threonine residues. The second sort of protein is a family of specialised activating proteins called cyclins that bind to CDK molecules and control their ability to phosphorylate targets. Cyclins themselves undergo a cycle of synthesis and degradation within each division of the cell cycle. There are a variety of species of cyclin, e.g. cyclin A and cyclin B.

Chao Y et al (1998) Cancer Research 58: 985-990 report a correlation between over-expression of cyclin A in patients and proliferative activity of tumour cells compared to those patients expressing a normal cyclin A level. Patients over-expressing cyclin A had a shorter median disease-free survival time than those who did not over-express. Chao et al (1998) also report that a cyclin A-interacting protein (Skp 2) did not exhibit the same correlation with tumour cell activity as cyclin A when over-expressed. Chao et al (1998) remark on how expression of Skp 2 appears to be involved in the control of cell cycle progression but caution that the actual biochemical function of Skp 2 is still not known.

In a more recent paper, Chao Y et al (1999) Cancer Letters 139: 1-6 conclude that cyclin A may provide a useful target for the exploration of new anti-hepatocelluar carcinoma (HCC) therapeutics. In particular, Chao et al (1999) showed that an over-expression of cyclin A in HCC cells could be inhibited with antisense mRNA for the cyclin A gene. Although an over-expression of Skp 2 is apparently also associated with HCC cell proliferation, Chao et al (1999) indicate that the biochemical function of Skp 2 remains unknown. For example, the results of an experiment seeking to block over-expression of Skp 2 using antisense mRNA suggests that abnormal Skp 2 expression has no direct correlation with HCC proliferation.

The activity of CDK is subject to regulation in the cell and a CDK inhibitor protein (p27) has been identified. In normal cells p27 has been shown to regulate the action of CDK's that are necessary for DNA replication. Levels of p27 are found to be high in quiescent cells and low in cells stimulated to divide. p27 appears to act as a brake on cell division by inhibiting activated CDK which itself drives cells to divide. A reduction in the level of p27 frees activated CDK from inhibition and drives cells to divide. Consistent with this activity of p27 is the way in which its destabilisation correlates generally with tumour aggressiveness and poor prognosis for cancer patients.

The cell cycle control system is a dynamic system and p27 itself does not remain at a constant level in the cell. The level is different depending on the point in the cell cycle. Lower levels of p27 arise due to breakdown via ubiquitination and subsequent proteasome-mediated degradation. A requirement for ubiquitin-mediated degradation of p27 is phosphorylation of the threonine residue 187 (T187) by activated CDK. The enzymes needed for ubiquitination of phosphorylated p27 are not known, although from knowledge of ubiquitination in systems such as yeast it is expected that there may be a human ubiquitin-protein ligase (E3) specific for p27.

Sutterlüty H et al (1999) Nature Cell Biology 1: 207-214 report that Skp 2 promotes the degradation of p27 in cells via the ubiquitination pathway. Skp 2 is a protein member of the F-Box-Protein (FBP) family. Skp 2 appears to be a p27 specific receptor of a Skp 1, CulA (Cdc53), F-Box Protein (SCF) complex. Such complexes are known in yeast and act as ubiquitin-protein ligases (E3) in which the FBP subunit has specificity for the substrate for ubiquitination. E3 facilitates the transfer of an activated ubiquitin molecule from a ubiquitin-conjugating enzyme (E2) to the substrate to be degraded. Similarly, in humans there are SCF complexes and Skp 2 is an FBP which has an ability to interact specifically with p27 and which appears to be essential in the ubiquitin-mediated degradation of p27. Both in vivo and in vitro, Skp 2 is found to be a rate-limiting component of the cellular machinery which ubiquitinates and degrades phosphorylated p27.

Skp 2 appears to be the product of a single gene and as such has an unusual ability in that it is able to drive cells to divide. This ability is shared with only a few other known gene products, e.g. E2F-1, c-Myc and cyclin E-CDK2 complexes. Timely accumulation of Skp 2 at the G1/S transition of the cell cycle may be one of the few rate-limiting steps controlling the initiation of DNA replication in mammalian cells. Sutterlüty et al (1999) found that a mutant of Skp 2 which does not assemble into an SCF complex was defective in promoting the elimination of ectopically produced wild-type p27. Also, mutant Skp 2 produced an activation of cyclin-E/A associated kinases and an induction of the S phase. Skp 2 also appears to have an independent binding site for CDK and activated CDK is involved in the phosphorylation of the T187 residue of p27. Sutterlüty et al (1999) also note how normal Skp 2 induces an accumulation of cyclin A protein, even when activation of cyclin-E/A-dependent kinases and entry into S phase are blocked by the expression of a non-degradable p27 mutant. What is concluded is that Skp 2 up-regulates cyclin A and independently of this down-regulates p27. The mechanism by which Skp 2 up-regulates cyclin A is not known. There is a suggestion that observed increased levels of Skp 2 in transformed cells might contribute to the process of tumourigenesis, at least partly, by causing an increase in the rate of degradation of the tumour suppressing agent p27. A lack of p27 expression correlates with a reduced disease-free survival of patients with colorectal and breast cancer. Also, p27 has been found to be haplo-insufficient for tumour suppression.

Carrano A. C. et al (1999) Nature Cell Biology 1: 193-199 report how Skp 2 interacts physically with phosphorylated p27 both in vitro and in vivo. Whilst every component of the ligase machinery required for p27 ubiquitination remains to be discovered, Carrano et al (1999) demonstrate that Skp 2 is a critical part of this machinery and provides substrate recognition and specificity for p27. Antisense oligonucleotides against Skp 2 were found to decrease Skp 2 expression in cells and thereby result in increased levels of endogenous p27. Carrano et al (1999) also confirm an additional need for cyclin E-CDK 2 or cyclin A-CDK 2 for ubiquitination of p27 to take place. p27 degradation in cells appears to be subject to dual control by accumulation of both Skp 2 and cyclins following mitogenic stimulation.

Of interest to scientists elucidating the molecular bases of cancer is a field of study relating to the molecular basis of the control of gene expression. Previously unconnected with the apparently essential roles of Skp 2 and p27 in cancer is the protein Pontin 52 reported in Bauer A. et al (1998) Proc. Natl. Acad. Sci. USA 95: 14787-14792. Pontin 52 is a nuclear protein which has a binding site for the TATA box binding protein (TBP) and β-catenin. A protein equivalent to Pontin 52 is found in rats and is called TIP49. Wood M. A. et al (2000) Molecular Cell 5: 321-330 observe that c-Myc oncogenic transformation of cultured rat embryo fibroblasts required TIP49 as an essential co-factor.

It is thought that RNA polymerase subunits might provide targets for transcriptional regulators in controlling gene expression. The RNA polymerase II subunit 5 (RPB5) has been demonstrated to interact with RPB5-mediating protein (RMP), a protein that inhibits the trans-activation function of Hbx when overexpressed (Dorjsuren D. et al (1998) Molecular Cell Biol. 18: 7546-7555).

An RMP homologue, NNX3, has also been referred to in the literature (Van Leuven et al. (1998) Genomics 54: 511-520), and was found to be expressed in skeletal muscle, ganglia and tumour cell lines. However, its function was not elucidated.

Genebank sequence AC008507.6 and AC073732 provide human and mouse contigs, respectively, which have regions of homology to the corresponding RMP/NNX3 coding sequences.

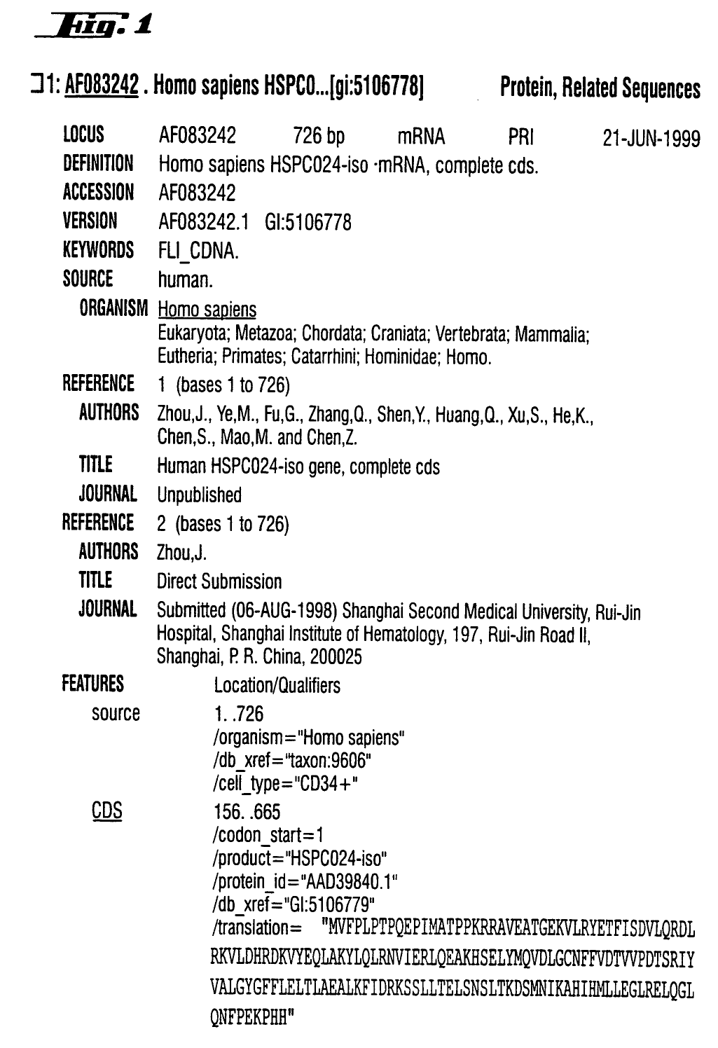

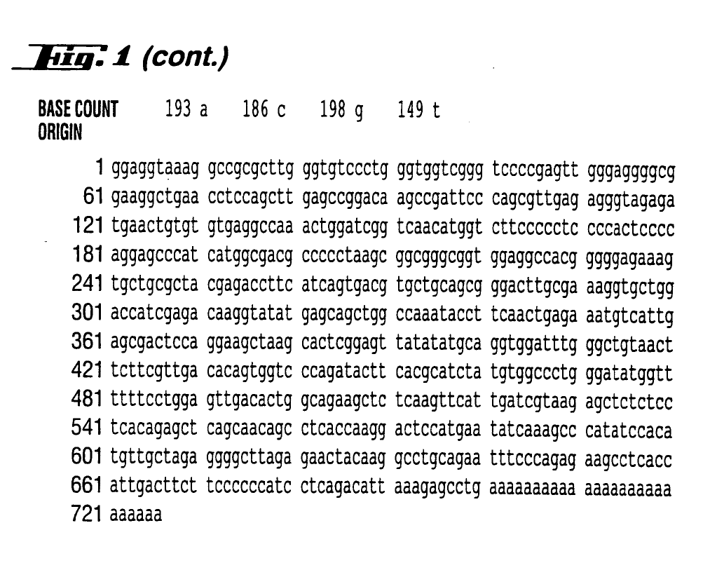

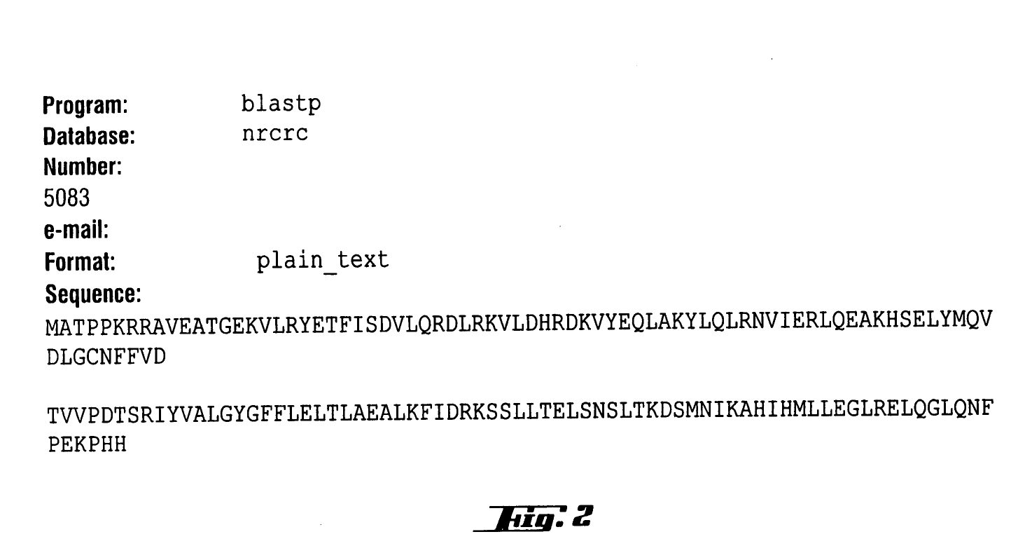

Genebank sequence AF083242 comprises 726 base pairs and is shown in FIG. 1 as SEQ ID NO: 2. The DNA sequence is not known to encode any known structural or functional protein, nor is the sequence known to have any regulatory or other effects on the genome. Also available is an amino acid sequence derived from the cDNA sequence. This is set forth as SEQ ID NO: 1 in FIG. 2. There are also a number of expressed sequence tags (ESTs) having varying degrees of homology with the Genebank sequence (SEQ ID NO: 2) shown in FIG. 1.

### SUMMARY OF THE INVENTION

The present invention provides a protein or peptide having at least 5 consecutive amino acids of:

MEAPTVETPP DPSPPSAPAP ALVPL (SEQ ID No. 3),

preferably at least 10, 15, 20 or more consecutive amino acids of SEQ ID NO: 3. More preferably, the peptide sequence is N-terminal to any additional peptide or protein sequence. Most preferably, the present invention provides a protein having the sequence as set forth in SEQ ID NO: 4. Optionally the protein or peptides may be phosphorylated.

Antibodies that specifically recognize the protein or peptide of the invention are provided, in particular phosphospecific antibodies.

Also provided are nucleic acids encoding the protein or peptides of the present invention or their complementary sequences, including antisense and sense oligonucleotides that optionally may include nucleotide residues that are resistant to nuclease degradation, such as phophorothioates and/or methylphosphonates.

Also provided are nucleic acid constructs comprising the nucleic acids of the invention, as well as host cells containing the constructs, in particular eukaryotic host cells, preferably an insect cell or a mammalian cell.

The present invention also encompasses the use of an antibody, nucleic acid, construct or host cells of the invention for the manufacture of a medicament for the prophylaxis or treatment of cancer, as well as for use as a pharmaceutical.

In another aspect of the invention, a complex comprising RMP or a fragment thereof and at least one other protein other than RPB5 is provided. Preferably, the complex comprises Skp2, STAP1, TIP48, TIP49, or prefoldin, optionally together with RPB5.

In a further aspect of the invention, various methods of diagnosing a condition dependent on amino acid regulation are provided. In one embodiment, the method comprises the steps of:

(a) collecting a sample from an individual;

(b) analyzing RMP or RMP activity of the sample; and

(c) correlating the presence of RMP or RMP activity in the sample with a condition dependent on amino acid regulation.

Conditions dependent on amino acid regulation include chronic diseases such as chronic renal, cardiac, hepatic, or pulmonary diseases, cancer, AIDS dietary conditions, and conditions sensitive to rapamycin treatment. The method may include determining the phosphorylation state of the RMP and correlating the presence of phosphorylated RMP with the condition. Alternatively, the level of RMP in a sample can be determined and compared to a standard level; whereby a decrease in RMP level relative to the standard level can be correlated with a condition dependent on amino acid regulation. The cellular localization of RMP in a cell can also be used as an indicator of the condition. In a further aspect, RMP activity is determined, for example, by analysing induction of (or lifting suppression of) expression of at least one gene that results in amino acid biosynthesis.

Also provided is a method of selecting individuals for treatment with rapamycin based on the diagnostic methods of the invention.

Kits comprising various reagents useful in the diagnostic methods of the invention are also provided, such as kits comprising one or more of an antibody that recognizes RMP; in particular a phosphospecific antibody, a means for collecting a sample, means of detecting an RMP-specific antibody, optionally together with instructions, a cell lysis buffer, an assay buffer, a secondary antibody and other reagents or receptacles useful for carrying out the diagnostic assays.

Also provided by the invention are various methods of screening for a modulator of RMP degradation (or identifying agents effective against conditions dependent on amino acid regulation in the same way). For example, a method is provided comprising the steps of:

(a) incubating a cell extract comprising RMP in a reaction mixture comprising a potential modulator of RMP degradation

(b) determining the amount of said RMP or phosphorylated RMP, optionally also a control protein; and

(c) correlating the presence of a modulator of RMP degradation with a change in level of RMP or RMP phosphorylation in the reaction mixture, relative to when said potential modulator is absent from the reaction mixture. The modulator can be an RMP suppressor, in which case the change in level can be maintenance of unphosphorylated RMP levels. Alternatively, the modulator can be an RMP activator, in which case the change in level can be an increase in RMP phosphorylation or a decrease in RMP level.

A method for identifying anti-cancer compounds is also provided by the invention, comprising measuring the binding of RMP to RPB 5 (RNA pol II subunit 5; Cheong et al., EMBO J. 14 (1), 143-150 (1995)), in the presence of a test compound, optionally also measuring the binding in the absence of a test compound.

In yet another embodiment, a method of identifying an anti-cancer agent is provided comprising contacting an RMP protein complex with a test compound and then determining one or more of: (a) the amount of intact complex remaining, (b) the amount of intact complex lost, or (c) the amount(s) of free protein or polypeptide subunit(s) released from the complex. Optionally, the method may comprise the step of forming the complex from its protein subunit components prior to contact with the test compound.

Also encompassed by the invention are anti-cancer agents or modulators of RMP degradation, other than rapamycin, identified by any of the methods of the present invention method, preferably an anti-proliferative agents.

In another aspect the invention provides the use of an agent, other than rapamycin, identified by a screening method of the invention as a pharmaceutical and/or for the manufacture of a medicament for the prophylaxis or treatment of cancer.

In a further aspect the invention provides a method of inhibiting a condition dependent on amino acid regulation comprising administering an effective amount of a compound, other than rapamycin, identified by a screening method of the invention. Such conditions include, without limitation, cancer, angiogenesis, diabetes, cell aging, neurodegenerative disease, immunological disorders, muscle degeneration, stress (trauma, thermal bum, sepsis, fever), chronic diseases (chronic renal, cardiac, hepatic, and pulmonary diseases) and AIDS.

### DETAILED DESCRIPTION OF THE INVENTION

The inventors have screened a variety of different cancer cell types for levels of expressed Skp 2 and p27. The inventors have also carried out co-transformation of primary rodent fibroblasts with both Skp 2 and H-RAS^{G12V}. Out of these experiments the inventors have discovered that Skp 2 is an oncogene responsible for many human cancers.

In exploring the oncogenic function of Skp 2 the inventors have unexpectedly discovered a novel protein called Skp 2-associated protein one (STAP1). The inventors generated antibodies against STAP1 and used these antibodies to immunoprecipitate STAP1 from HeLa cells. The immunoprecipitates were surprisingly found to contain several STAP1-co-immunoprecipitating proteins. The proteins including STAP1 were found to form a complex. The molecular weights of proteins were determined by mass spectrometry and then databases of proteins and gene sequences were searched to try and identify the proteins. Quite unexpectedly the STAP1-containing complex of proteins is found to include TIP48, TIP49, RPB 5 (RNA pol II subunit 5), RMP1 (RNA pol II subunit 5 mediating protein) as well as other hitherto unknown proteins.

Without wishing to be bound by any particular theory, the inventors have realised that Skp 2 represents an oncogene which can interact through STAP1 and its complex with various elements of a transcriptional control apparatus, such as TIP49 (and TIP48), RPB5 and RMP1, and these links provide new points of attack for inhibitors of protein-protein binding and enzymic activities. Such inhibitors are expected to have anti-proliferative and therefore anti-cancer properties. In the light of these discoveries, suitable screening assays can now be developed to identify new anti-cancer agents, for example.

In one aspect of the invention, an RMP1 coding sequence is provided as well as the resulting protein product. Previously published sequences of human RMP1 homologues lack about 70 amino acids from the N-terminal of the sequence provided in the present invention (NNX3; Van Leuven et al.; NCBI Accession No. AAD08679, 21 Jan. 1999) or replaced about 50 amino acids from the N-terminal sequence of RMP1 with about 25 amino acids from an unrelated sequence (Dorjsuren et al.; NCBI accession No. NP_{—}003787, Nov. 1, 2000). Now that the human genome sequence has been elucidated, one might expect that it would be a routine matter to determine the RMP1 sequence provided by the present invention. Nevertheless, updated sequence databases still only provide the truncated human RMP1 homologous sequence (NCBI Accession No. XP_{—}012802, Apr. 16, 2001).

Thus in one aspect of the invention, a protein or peptide is provided comprising at least 5 consecutive amino acids of:

MEAPTVETPP DPSPPSAPAP ALVPL (SEQ ID No. 3),

preferably at least 10, 15, 20 or more consecutive amino acids of SEQ ID NO: 3. More preferably, the peptide sequence is N-terminal to any additional peptide or protein sequence. Most preferably, the present invention provides a protein having the sequence as set forth in SEQ ID NO: 4. Optionally the protein or peptides may be phosphorylated, in particular the protein or peptide may comprise phosphoserine or phosphothreonine residues. Phosphopeptides are particularly useful for producing phosphospecific peptides, which can then be tested for reactivity with naturally occurring phospho-RMP. The proteins an peptides of the invention are particularly useful for diagnostic and screening methods described in more detail below.

Useful in the methods of the invention are also RMP1 variants or fragments thereof. RMP1 variants comprise sequences substantially homologous to SEQ ID NO: 4, particularly a degree of identity (homology) of at least 60%, preferably at least 70 or 80%, more preferably at least 90%, even more preferably 95%, most preferably at least 99%.

The RMP1 variants preferably have binding affinity for, and/or association affinity with, at least one transcription regulatory factor, optionally other proteins or polypeptides of the cell. Preferred RMP1 variants have binding affinity for, and/or association affinity with, one or more of TIP48, TIP49 and RPB5, optionally also with other proteins or polypeptides.

Variants of the RMP1 include all forms of mutant variants, for example wherein at least one amino acid is deleted or substituted. Any changes involving substitution of amino acids are preferably neutral or conservative substitutions.

Other variants include proteins or polypeptides comprising at least one additional amino acid in the sequence, and/or further comprising an additional amino acid sequence or domain, such as fusion proteins, as is well known in the art.

Further variants of the RMP1 proteins or polypeptides include those wherein at least one of the amino acids in the sequence is a natural or unnatural analogue. Also, one or more amino acids in the sequence may be chemically modified, e.g. to increase physical stability or to lower susceptibility to enzymic, particularly protease or kinase, activity.

In a further aspect of the invention, nucleic acids encoding the protein or peptides of the invention, as well as their complementary sequences, are provided. In particular, the nucleic acid comprises at least a fragment of the sequence SEQ ID NO: 5, (which encodes the amino acid sequence SEQ ID NO: 3, and is found as a fragment of SEQ ID NO: 6), or its complement, preferably comprising the sequence as set forth in SEQ ID NO: 6, or its complement. Such nucleic acids are useful as probes and primers, in particular to obtain the full length nucleic acid coding sequence of RMP1.

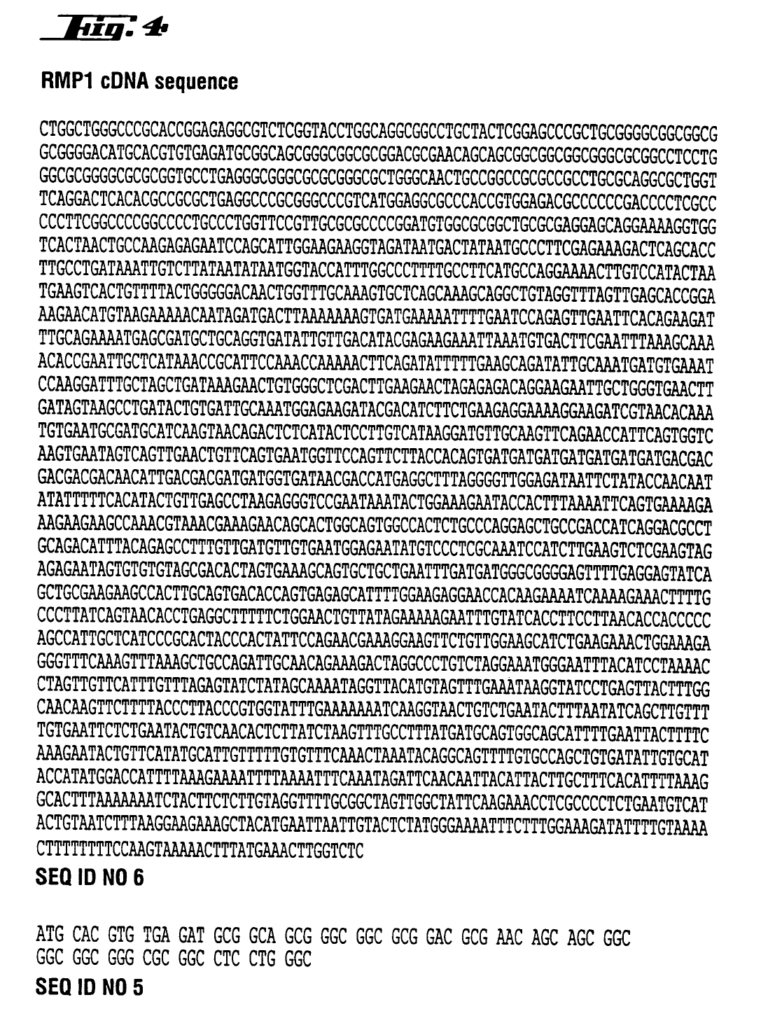

The RMP1 protein of the invention, or polypeptide fragments thereof, is encoded by a nucleic acid sequence substantially as set forth in FIG. 4 (SEQ ID NO: 6), or a sequence having at least 70% homology therewith, that is capable of hybridizing under low stringency conditions thereto, and comprising at least a fragment of SEQ ID NO.5, preferably a fragment of at least 5 or 10 nucleotides. Low stringency conditions employs around 0.01×SSC buffer compared to high stringency which employs about a 10 fold greater concentration. The sequence homology may be at least 80%, preferably at least 90%, more preferably at least 95%, even more preferably 97.5%, most preferably at least 99%.

In another aspect the invention provides a nucleic acid antisense to all or a part of a nucleic acid of SEQ ID NO: 5 or SEQ ID NO: 6, or antisense to a sequence having at least 70% homology with SEQ ID NO: 5 or SEQ ID NO: 6, that is able to hybridize under low stringency conditions to SEQ ID NO: 5 or SEQ ID NO: 6, and which encodes an RMP1 protein or polypeptide as hereinbefore described. Alternatively, the antisense RNA may be antisense to regulatory sequences of the RMP1 gene, in particular to 5′ upstream sequences (promoter region) of the gene. The sequence to which the nucleic acid is antisense to may have at least 80% homology (identity) with the reference sequence, preferably at least 90%, more preferably at least 95%, even more preferably at least 95%, most preferably at least 99%.

Nucleic acids are preferably to be at least 10 bases long, more preferably at least 15 even more preferably at least 50 bases long. The nucleic acids can be RNA or DNA, sense or antisense, and in some embodiments, double stranded or single stranded. In certain embodiments at least some of the nucleotide residues of the nucleic acids (sense or antisense) may be made resistant to nuclease degradation and these can be selected from residues such as phophorothioates and/or methylphosphonates.

The nucleic acids as hereinbefore described can advantageously be used as pharmaceuticals, preferred pharmaceutical applications being for the manufacture of a medicament for the prophylaxis or treatment of conditions dependent on amino acid regulation, such as cancer.

Thus, the invention also provides a method of preventing or treating a condition dependent on amino acid regulation, comprising administering to an individual an effective amount of a nucleic acid, preferably antisense nucleic acid, as hereinbefore described.

Other embodiments of the invention include nucleic acid constructs comprising (a) at least one nucleic acid sequence portion encoding an RMP1 protein or polypeptide of the invention (b) antisense nucleic acids as hereinbefore described (or their complement, for example, if expression of the antisense RNA in a ceil is foreseen), or (c) nucleic acids as hereinbefore described and at least one nucleic acid sequence encoding a protein other than RMP (or its homologues). Such constructs are not naturally occurring sequences. The constructs lack essential sequences of DNA which might permit them to function as vectors but are not naturally occurring as “hybrid” nucleic acids. They may include nucleic acid sequences that function as linkers or restriction sites. Preferred constructs are synthesised using methods of oligonucleotides synthesis well known to those of skill in the art.

Also provided are vectors comprising a construct as hereinbefore described. Preferred vectors are expression vectors, preferably plasmids or viruses although cloning vectors are also provided for, optionally in the form of plasmids.

The invention provides host cells containing vectors; preferably the host cell expresses an RMP1 protein or polypeptide of the invention. Preferred host cells are eukaryotic cells, more preferably insect cells or mammalian cells.

Constructs, vectors and transformed host cells of the invention are of use as pharmaceuticals, as well as for the manufacture of a medicament for the prophylaxis or treatment of a condition dependent on amino acid regulation.

Antibodies that specifically recognize the protein or peptide of the invention are also provided, in particular phosphospecific antibodies. Methods for producing antibodies are well known in the art. An antibody specific for RMP can be easily obtained by immunizing an animal with an immunogenic amount of RMP. Therefore, an antibody recognizing RMP embraces either of polyclonal antibody and antiserum which are obtained by immunizing an animal, and which can be confirmed to recognize RMP of this invention by Western blotting, ELISA, immunostaining or other routine procedure known in the art.

A part of RMP, optionally linked to a carrier protein, such as a bovine serum, can also be used as the immunogen, in particular where phosphospecific antibodies are desired. The protein fragment may be synthesized by a peptide synthesizer and preferably comprises 8 or more amino acids.

It is well known that if a polyclonal antibody can be obtained by sensitization, a monoclonal antibody is secreted by the hybridoma, which may be obtained from the lymphocytes of the sensitized animal (Chapter 6, Antibodies A Laboratory Manual, Cold Spring Harbor Laboratory Press,1988). Therefore, monoclonal antibodies recognizing RMP, in particular phosphospecific antibodies, are also provided. By “phosphospecific antibody”, it is meant that the antibody specifically recognises a phosphopeptide fragment of RMP or phosphorylated RMP, but does not recognise unphosphorylated RMP or peptide fragments thereof.

In this invention, an antibody also embraces an active fragment thereof. An active fragment means a fragment of an antibody having activity of antigen-antibody reaction. Specifically named, these are active fragments, such as F(ab′)2, Fab′, Fab, and Fv. For example, F(ab′)2 results if the antibody of this invention is digested with pepsin, and Fab results if digested with papain. Fab′ results if F(ab′)2 is reduced with a reagent such as 2-mercaptoethanol and alkylated with monoiodoacetic acid. Fv is a mono active fragment where the variable region of heavy chain and the variable region of light chain are connected with a linker. A chimeric antibody is obtained by conserving these active fragments and substituting the fragments of another animal for the fragments other than these active fragments. In particular, humanized antibodies are envisioned.

Methods for detecting RMP embrace, for example, the use of an antibody as referred to above, optionally with the use of an enzyme reaction. The antibody recognizing RMP can be detected using secondary antibodies specific for the RMP antibody, which are optionally labelled with a radiolabel, an enzyme, avidin or biotin, or fluorescent materials (FITC or rhodamine), for example.

Also encompassed by the invention is the use of an antibody that specifically recognizes RMP for the manufacture of a medicament, in particular a medicament for the prophylaxis or treatment of cancer, or as a pharmaceutical.

In a further aspect of the invention, a complex comprising RMP1, a variant or fragment thereof and at least one other protein other than RPB5 is provided. The RMP1 complex preferably comprises one or more of Skp2, STAP1 (FIG. 2; SEQ ID NO: 1), TIP48, TIP49 and prefoldin, optionally together with RPB5. The subunits STAP1, TIP48, TIP49, RPB 5, RMP 1 may be present in a ratio of about 1:1:1:1:1, although other ratios are possible.

Optionally, the additional proteins or polypeptides may also be in a stoichiometric ratio of 1:1, but again other ratios are possible.

The invention also provides a transcription regulatory protein complex comprising RMPI, a variant or fragment thereof and three or more other proteins or polypeptides. These other proteins or polypeptides may be as described above.

In any of the complexes of the invention hereinbefore described the constituent protein or polypeptide subunits may each have a molecular weight in the range 5 to 500 kD, preferably 5 to 300 kD, more preferably, 10 to 200 kD, even more preferably 10 to 100 kD. SDS-PAGE or mass spectrometry provide ways of establishing molecular weights.

Complexes of the invention as hereinbetore described may be obtainable by immunoprecipitation using an antibody reactive against STAP1 or RMP1, for example. Ideally, complexes of the invention are substantially free of other cellular contaminants. Thus, isolated complexes may be of at least 80% purity, preferably 90% purity, more preferably 95% purity, even more preferably 99% purity. Purity can be determined by various methods, e.g. SDS-PAGE.

Alternative ways of producing complexes of the invention may be to assemble them from constituent protein or polypeptide subunits. One way is to have a cell transformed to overexpress each of the constituent subunits so that assembly of the complex takes place in the cell. A preferred expression system employs transformed insect cells.

Another way is to mix the constituent subunits together in vitro under conditions sufficient for self-assembly of the complex. Preferably, the mixing of subunits occurs substantially simultaneously. There are many other possibilities of mixing including assembly of partial complexes in transformed cells followed by isolating and mixing them with the remaining subunits in vitro under conditions promoting self assembly of the whole complex. Also, partial complexes can be made in vitro by mixing and then mixed with the remaining subunits. The order of mixing subunits or partial complexes in vitro is not believed to be critical in order to yield complexes.

In a further aspect of the invention, methods of diagnosing a condition dependent on amino acid regulation are provided. In its broadest aspect, the method comprises the steps of:

(a) obtaining a sample from an individual;

(b) analyzing RMP or RMP activity of the sample; and

(c) correlating the presence of RMP or RMP activity in the sample with a condition dependent on amino acid regulation.

The methods of the present invention will typically involve the determination of the presence, level, cellular localization or activity of RMP in a cell or tissue sample, which sample will often be obtained from a human, but one can also readily understand that samples tested by the present method can be obtained from agriculturally important mammals, such as cattle, horses, sheep, etc., or other animals of veterinary interest, such as cats and dogs. The assay can be carried out on any cell or tissue sample, such as somatic tissues, germline tissues, or cancerous tissues, as well as on samples from body fluids, such as pleural fluid, blood, serum, plasma and urine.

A “sample” is the material being analyzed which is usually, but not necessarily, subjected to pretreatment to provide the RMP in assayable form. This would normally entail forming a cell extract, methods for which are known in the art (for example, see Scopes, Protein Purification: Principles and Practice, Second Edition (Springer-Verlag, N.Y., 1987)).

In the broader aspects of the invention, there is no limitation on the collection and handling of samples as long as consistency is maintained. The sample is obtained by methods known in the art, such as, biopsies, surgical resections, smears, or the like.

Consistency of measurement of RMP or RMP activity in clinical samples can be ensured by using a variety of techniques. For example, to control for the quality of each tissue extract, another enzymatic activity, such as alkaline phosphatase, can serve as an internal control. In addition, an internal standard can be measured concurrently with RMP in the sample as a control for assay conditions. Thus, the analyzing step can comprise detecting a control protein in the sample, optionally normalizing the value obtained for RMP with a signal obtained with the control protein.

The presence of RMP in the sample can be determined by detecting the RMP protein using methods known in the art. In this invention, there are no limitations on the type of assay used to measure RMP or RMP activity. For example, RMP can be detected by immunoassays using antibodies specific for RMP. The antibody can be used, for example, in Western blots of two dimensional gels where the protein is identified by enzyme linked immunoassay or in dot blot (Antibody Sandwich) assays of total cellular protein, or partially purified protein. In preferred embodiments the presence of phosphorylated RMP is determined using a phosphospecific antibody.

Methods for sample concentration and protein purification are described in the literature (see Scopes, 1987). For example, if desired, the RMP present in the cell extract can be concentrated, by precipitating with ammonium sulfate or by passing the extract through a commercially available protein concentration filter, e.g., an Amicon or Millipore, ultrafiltration unit. The extract can be applied to a suitable purification matrix, such as an anion or a cation exchange resin, or a gel filtration matrix, or subjected to preparative gel electrophoresis. In such cases, the RMP and protein yield after each purification step needs to be considered in determining the amount of RMP in a sample. However, because these separations are generally difficult and may result in loss of RMP, cell extracts that have not been pretreated are preferred for the assay.

As described above, in one embodiment, the presence of RMP in a sample is determined using immunological techniques. The presence of RMP, in particular the RMP1 of the present invention can provide useful information for diagnosing a condition dependent on amino acid regulation. In a preferred embodiment, the phosphorylation state of the RMP is determined, for example by using phosphospecific antibodies in immuno “dot” blots of the sample, such as cell extracts or pretreated extracts. The presence of phosphorylated RMP in the sample is indicative of a condition dependent on amino acid regulation.

In an alternative embodiment, the level of RMP in a sample is determined and compared with a standard level. A decrease in RMP level relative to said standard level is indicative of a condition dependent on amino acid regulation. The standard level can be the level of RMP in a sample obtained from a second individual unaffected with the condition. If the condition is cancer, then the standard level can be the level of RMP in a non-cancerous tissue obtained from the same individual or the level of RMP in a sample removed from tissue adjacent to a cancerous growth.

The standard value of RMP is selected to divide a population of patients into two statistically significant classes, those having a condition dependent on amino acid regulation and those not having that condition. In one embodiment of the invention, the standard value is selected to be the level of RMP found in a tissue known to have RMP, that is the level of RMP in normal tissue unaffected with the condition.

Alternatively, the standard level of RMP is determined by collecting data to obtain a statistically significant correlation of RMP levels with one or more conditions dependent on amino acid regulation, relative to normal individuals.

Typically, a change in RMP level between an affected and an unaffected individual would be at least two, preferably at least five, more preferably at least 10-fold under comparable conditions of measurement. One of ordinary skill in the art recognizes that if a less sensitive assay is chosen to determine the level of RMP in a sample, it may be necessary to increase the amount of sample/extract used or to pretreat the extract using routine methods as described above to provide detectable levels of RMP. The assay methods do not necessarily require measurement of absolute values of RMP, unless it is so desired, because relative values are sufficient for the methods of the present invention; however, any known method for quantitating RMP levels could be used for this determination.

A predetermined range of RMP levels diagnostic of a condition can be established for the same cell or tissue sample obtained from subjects having known clinical symptoms. Sufficient measurements are made to produce a statistically significant range of values for the value to which a comparison will be made. The predetermined range of RMP is typically obtained by using the same assay technique that will be used in the application of the method to an individual being tested to ensure the highest correlation. Standard values may vary with the specific cell or tissue extract for which RMP is measured and with the specific assay used. The predetermined range of RMP for a given cell or tissue sample can then be used to determine a standard value for the RMP level that would be considered as the value from an unaffected individual and correlated to a negative diagnosis. The method of the invention does not require the measurement of any other substance or, in this latter described aspect of the invention, can even be dependent upon a single measurement, once a standard level for an assay procedure is established. In the case of relying on a single measurement, the assay is preferably carried out on a sample under conditions that would quantitate the RMP level.

A measured low level of RMP (or no RMP) relative to the standard value (for example, and not meant to be considered limiting, no detectable activity in undiluted samples, detectable activity only in samples incubated for long lengths of time, etc.) is an indication of a condition dependent on amino acid regulation, suggesting that the physician should employ an appropriate therapy. An RMP level similar to or higher than the standard level is an indication of a favorable diagnosis.

Those of skill in the art will also recognize that, while the use of cell extracts is preferred for most purposes, one can also modify the method so that intact cells can be employed. In this embodiment, one treats intact cells with an antibody specific for RMP, for example, to visualize the cellular localization of the RMP using immunohistochemical techniques known in the art (Van Leuwen, 1998, Genomics 54:511-520). Alternatively, cellular localization can be determined analysing samples obtained from nuclear extracts and cytoplasmic extracts after fractionation of cellular compartments on sucrose gradients, for example. Although not wishing to be bound by theory, the present inventors believe that RMP is localized to the nucleus in normal tissues where it may inhibit expression of genes responsible for amino acid biosynthesis. Conversely, in affected tissues from an affected individual, RMP is believed to be released from the nucleus, lifting suppression of amino acid synthesis, and transfers to the cytoplasm with said condition. In preferred embodiments, the RMP localization is visualized with a phosphospecific antibody.

In a further embodiment, RMP activity is measured. In a preferred embodiment the RMP activity is the induction of expression (or removing suppression) of at least one gene that results in amino acid biosynthesis.

In a preferred format, DNA chip arrays are designed comprising various gene sequences encoding proteins involved in amino acid biosynthesis. These gene sequences are easily obtainable from various databases (e.g., Genebank, NCBI). RNA samples from individuals are tested to determine whether any amino acid biosynthetic gene has been upregulated compared to a standard value, determined essentially using the techniques described above. A sample shown to have a higher level of a specific RNA, in particular one that encodes proteins involved in amino acid biosynthesis, relative to a control value or control sample is indicative of a condition susceptible to amino acid regulation. Thus in this embodiment, RMP activity equates with its ability to repress expression of genes involved in amino acid biosynthesis. In this embodiment, any method of RNA preparation known in the art, or yet to be developed, may be employed but diligence should be used in maintaining the quality of RNA between samples, for example minimizing degradation.

No matter which diagnostic method is used, the condition dependent on amino acid regulation can be a chronic disease, such as chronic renal, cardiac, hepatic, or pulmonary diseases, cancer, such as a cancer selected from the group consisting of leukaemia, bladder cancer, neuroblastoma, gastric cancer, prostate cancer, breast cancer, colon cancer, renal cancer, ovarian cancer, liver cancer and lung cancer, with the proviso that the cancer is not Hodgkin's disease when the mere presence of RMP is determined, or defective immunological response, such as in AIDS, neurodegeneration, muscle degeneration, angiogenesis, diabetes, cell aging, or a dietary condition. Also encompassed are conditions that are susceptible to rapamycin treatment.

The diagnostic methods of the present invention allow physicians to administer an appropriate therapy. Assays for a given analyte, including these assays for RMP, are not expected to be obtained or to be interpreted by an attending physician in the absence of additional information. Although the present method for testing the RMP provides much useful information regarding the condition, tests that may provide additional information in conjunction with the present method may be used. Additionally, the results of any assay are best considered to be indicative of the probability of a presence of a clinical condition rather than as absolute proof. The same situation exists for the present invention. Nevertheless, an indication of diagnosis is clinically useful information and can be used by a skilled medical practitioner in combination with other information to care for patients in a more informed manner than would be possible if the information were not available. In particular, a physician can determine whether additional diagnostic tests quantitating RMP should be required periodically to follow the effect of therapy.

The effect of rapamycin on RMP phosphorylation further suggests that the diagnostic methods of the invention can be used to determine whether an individual will be sensitive to rapamycin treatment or not. Many individuals suffering from cancer do not respond to rapamycin and the ability of a physician to select patients that will or will not respond to a particular therapy is particularly useful. Thus, in a further aspect of the invention, a method is provided for selecting individuals for treatment of a condition dependent on amino acid regulation, in particular cancer, using any of the diagnostic methods referred to herein for treatment with rapamycin, or any other agent depending on IGF or TOR pathways

The present invention also provides kits for performing the methods of the invention. Such kits can be prepared from readily available materials and reagents and can come in a variety of embodiments. For example, such kits can comprise any one or more of the following materials: an antibody that recognizes RMP, preferably a phosphospecific antibody, a means for collecting a sample, such as a filter or other semi-porous support, reaction tubes, buffers (e.g., cell lysis buffer or an assay buffer), a secondary antibody,a means of detecting the RMP-specific antibody, enzyme substrates, control reagents, oligonucleotides, and instructions. A wide variety of kits and components can be prepared according to the present invention, depending upon the intended user of the kit and the particular needs of the user.

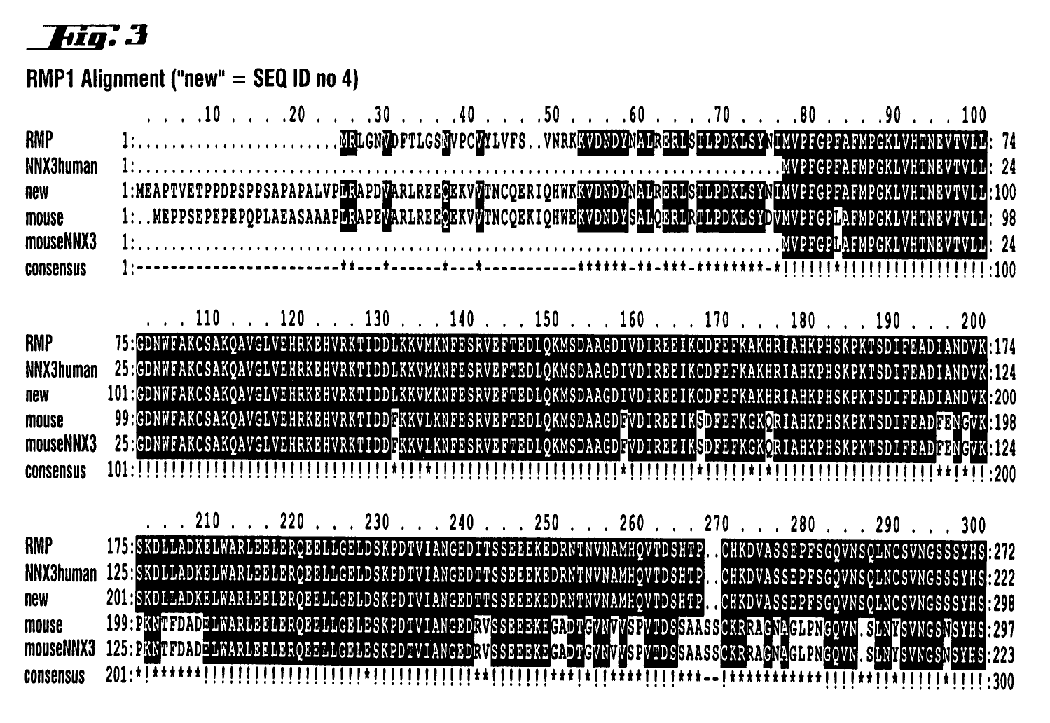

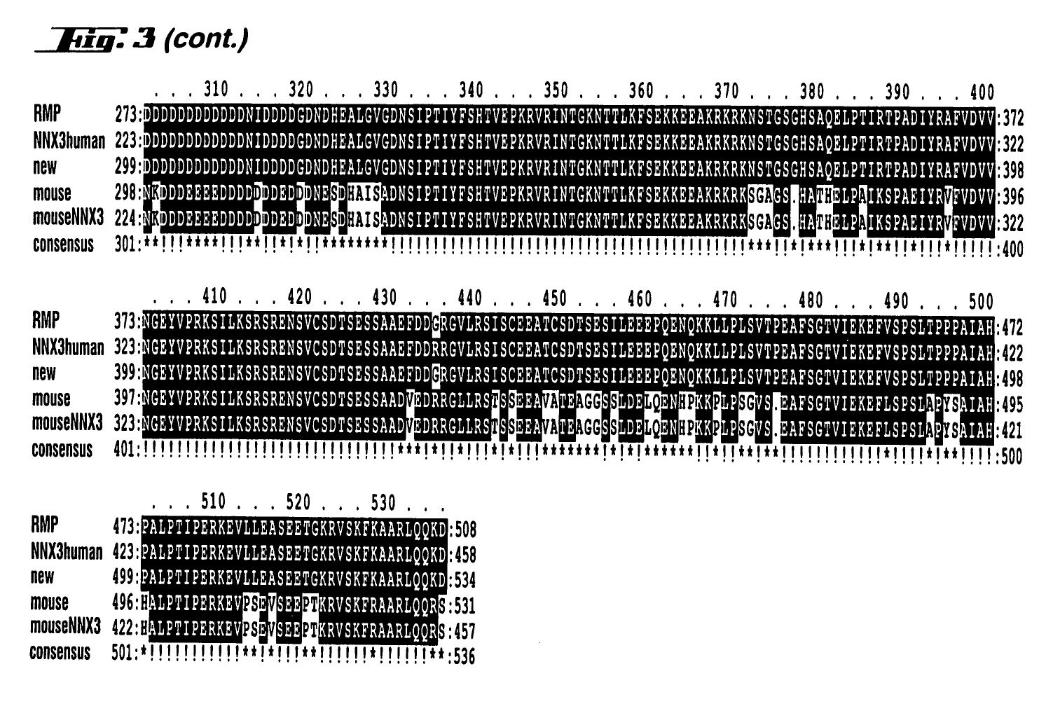

For clarification, the screening (and in some cases depending on the sample, diagnostic) methods of the invention can employ (or detect) RMP1 or any variant functional fragment or homologue, such as NNX3 or RMP known in the art. Thus, unless clear from the context, reference to RMP or RMP1 encompasses both variants of RMP1 and fragments thereof, although typically RMP, RMP1 or NNX3 are preferred. Preferably, RMP1 as set forth in FIG. 3 is used in the screening methods or detected in the diagnostic methods.

Thus, in another aspect the invention provides a method of identifying anti-cancer compounds comprising measuring the binding of a test compound to a RMP1 protein or polypeptide of the invention, optionally also comprising measuring the binding of a control compound to an RMP1 protein or polypeptide.

Also provided is a method of screening for an agent effective against conditions dependent on amino acid regulation,in particular cancer, said method comprising the steps of:

(a) incubating a cell extract comprising RMP in a reaction mixture comprising a potential modulator of RMP degradation;

(b) determining the amount of said RMP or phosphorylated RMP; and

(c) correlating the presence of a modulator of RMP degradation (i.e., an agent effective against conditions dependent on amino acid regulation) with a change in level of RMP or RMP phosphorylation in said reaction mixture, relative to when said potential modulator is absent from said reaction mixture.

In essence, the techniques described above for preparing cell extracts and determining RMP in diagnostic methods can be applied to the screening methods of the present invention. For example, the detecting step can comprise incubating the RMP with an antibody, preferably, a phosphospecific antibody. An effect of a compound on RMP degradation, phosphorylation, cellular levels, localization and RMP activity can be determined in screening for effective agents. Similarly, a control protein can be used to normalize values obtained for RMP detection.

The methods can be applied to identify RMP suppressors, in which case, for example, the change in level is maintenance of unphosphorylated RMP levels, or RMP activators, in which case, for example, the change in level is an increase in RMP phosphorylation or a decrease in the overall RMP level.

Also provided by the present invention is a method for identifying an agent effective against conditions dependent on amino acid regulation, in particular cancer, comprising measuring the binding of RMP to RBP5 in the presence of a test compound, optionally also measuring the binding in the absence of a test compound. The RMP is preferably as set forth in SEQ ID NO 4.

Preferred methods are solid phase assays and in most preferred embodiments the RMP1 protein, fragment or variant thereof is immobilised to the substrate. Most preferred substrates for ligand-binding assays are nickel or nickel coated, e.g. nickel coated microtiter plates, allowing the easy attachment of HIS6-tagged RMP1 to the solid surface. Naturally, other means of attachment can be easily applied. For immunoassays, filters that bind proteins, such as nitrocellulose or other suitable blotting materials are most preferred.

The protein and/or its binding partner (e.g., an antibody) may be labelled, for example with a fluorescent label, an enzyme label, biotin, a metal sol particle or a radiolabel. In preferred embodiments the label is europium.

In another aspect the invention provides a method of identifying an anti-cancer agent comprising contacting an amount of a complex as hereinbefore described with a test compound and then determining one or more of: (a) the amount of intact complex remaining, (b) the amount of intact complex lost, or (c) the amount(s) of free protein or polypeptide subunit(s) released from the complex.

The amount of complex may be determined by measuring one or more activities of the complex, preferably an enzymic and/or ligand binding activity. Where ligand binding is measured then the ligand may be selected from a nucleic acid or a protein, preferably the protein binding activity is an oncogene product. e.g. c-Myc or Skp 2 binding activity, beta-catenin binding activity, Hbx binding activity or RNA polymerase II binding activity. If an enzymic activity is measured then it may be ATPase activity, and/or DNA helicase activity.

In methods which determine the amount(s) of free protein or polypeptide subunits lost from the complex then the free protein or polypeptide subunit(s) may be one or more of RBP 5, RMP 1, TIP48, TIP49 or a STAP1 protein or polypeptide as hereinbefore described. Free protein or polypeptide subunit amounts may be determined by measuring an enzymic and/or ligand binding activity. When a ligand binding assay then the ligand may be selected from a nucleic acid or a protein, preferably the protein binding activity is an oncogene product e.g. c-Myc or Skp 2 binding activity, beta-catenin binding activity, Hbx binding activity or RNA polymerase II binding activity. When an enzymic activity is determined then it may be selected from one or more of ATPase activity or DNA helicase activity.

In all methods of anti-cancer agent screening there may be the further step of forming the complex from its protein subunit components prior to contact with the test compound.

Another aspect of the invention is the use of RMP1 as hereinbefore described in a method of screening, preferably any of the methods hereinbefore described. Allied to this aspect of the invention is the use of any one or more of RMP 1 for in vitro assembly of a complex as hereinbefore described.

As demonstrated below in the Examples, IGF and TOR pathways seem to converge on RMP, making RMP a target for many potential therapeutic agents. The invention permits the identification of agents effective against conditions regulated by amino acid synthesis, in particular anti-cancer agents, by performance of any of the methods of screening described herein. Preferred anti-cancer agents are those which inhibit proliferation of the cancer cells and which may be general anti-proliferative agents.

The invention includes all agents identified by performing the methods, other than rapamycin, and the use of these agents as pharmaceuticals, particularly as medicaments for the prophylaxis or treatment of cancer and other conditions regulated by amino acid biosynthesis.

Thus, in a further aspect the invention provides for the use of an agent (other than rapamycin) identified by a screening method of the invention as a pharmaceutical.

The invention further provides an agent identified by a screening method of the invention, for the manufacture of a medicament for the prophylaxis or treatment of a condition dependent on amino acid regulation, with the proviso that the agent is not rapamycin for use to treat cancer or an immunological defect.

The invention provides a method of preventing or treating a condition dependent on amino acid regulation comprising administering to an individual an effective amount of a construct, vector, host cell or antibody described above.

The invention also provides a method of inhibiting a condition dependent on amino acid regulation comprising administering an effective amount of the modulator identified by a screening method of the invention described above, other than rapamycin for the treatment of cancer or an immunological defect.

Where this specification refers to conditions dependent on amino acid regulation, it should be noted that these conditions exemplify one set of clinical condtions dependent on RMP. Thus, the diagnostic methods of the present invention can be used to diagnose any condition dependent on RMP. Similarly, the present invention provides screening methods for agents active against conditions dependent on RMP, the agents identified by the methods, as well as the use of those agents for conditions dependent on RMP, other than the known uses of rapamycin.

Also provided by the invention are the agents referred to above in a pharmaceutical composition, possibly in the presence of suitable excipients known to the skilled man. The compositions may be administered in the form of any suitable composition as detailed below by any suitable method of administration within the knowledge of a skilled man. The preferred route of administration is parenterally. In parenteral administration, the compositions of this invention will be formulated in a unit dosage injectable form such as a solution, suspension or emulsion, in association with a pharmaceutically acceptable excipient. Such excipients are inherently nontoxic and nontherapeutic. Examples of such excipients are saline, Ringer's solution, dextrose solution and Hank's solution. Nonaqueous excipients such as fixed oils and ethyl oleate may also be used. A preferred excipient is 5% dextrose in saline. The excipient may contain minor amounts of additives such as substances that enhance isotonicity and chemical stability, including buffers and preservatives.

Any protein is administered at a concentration that is therapeutically effective to prevent allograft rejection, GVHD, allergy and autoimmune diseases. The dosage and mode of administration will depend on the individual. Generally, the compositions are administered so that the functional protein is given at a dose between 1 pg/kg and 10 mg/kg, more preferably between 10 ug/kg and 5 mg/kg, most preferably between 0.1 and 2 mg/kg. Preferably, it is given as a bolus dose. Continuous short time infusion (during 30 minutes) may also be used. The compositions according to the invention may be infused at a dose between 5 and 20 μg/kg/minute, more preferably between 7 and 15 μg/kg/minute.

According to a specific case, the “therapeutically effective amount” of a composition needed should be determined as being the amount sufficient to cure the patient in need of treatment or at least to partially arrest the disease and its complications. Amounts effective for such use will depend on the severity of the disease and the general state of the patient's health. Single or multiple administrations may be required depending on the dosage and frequency as required and tolerated by the patient.

Preferred embodiments of the invention will now be described by way of example and where convenient with reference to drawings in which:

FIG. 1 shows a nucleotide sequence of STAP1 (SEQ ID NO: 2).

FIG. 2 shows a derived amino acid sequence of STAP1 (SEQ ID NO: 1).

FIG. 3 shows the amino acid sequence of RMP1 (“new”; SEQ ID NO: 4) aligned with previously published sequences.

FIG. 4 shows a nucleotide sequence encoding the first 25 amino acids ofRMP1 (SEQ ID NO: 5). And a nucleotide sequence encoding RMP1 (SEQ ID NO: 6).

### EXAMPLE 1

### Skp 2 and H-Ras^{(G12V)} Transfection of Cells Transforms Them

Skp 2 co-operates with H-Ras^{G12V} to cause cellular transformation of primary rodent fibroblasts as scored by colony formation in soft agar and tumour formation in nude mice. Such transformants express significantly lower levels of p27 than normal fibroblasts or E1A/H-Ras^{G12V}-transformed derivatives.

A sensitive assay of functional properties of candidate oncogenes derives from the use of embryo cell cultures that can be transfected with these genes singly or in combination. When introduced into rat embryo fibroblasts, oncogenes such as E1A or E2F1 are able to transform them only in the presence of a co-introduced, collaborating oncogene like the oncogenic version of H-Ras in which Gly¹² was changed to Val (G12V). Mammalian expression plasmids encoding Skp 2 and H-Ras^{G12V} were transfected either alone or in combination into primary rat embryo fibroblasts (REFs). After selection in G418 for 3 weeks, plates were scored for the presence of morphologically transformed colonies. In the absence of H-Ras^{G12V}, Skp 2 alone failed to give rise to morphologically transformed foci. In contrast, addition to H-Ras^{G12V} together with the Skp 2 gene gave rise to substantially increased number of morphologically transformed colonies, ranging on average from 70-110 colonies pre plate. Colonies produced by transfection of Skp 2 and H-Ras^{G12V} were easily established and gave rise to cell lines that grew rapidly in culture. These Skp 2/H-Ras^{G12V}-expressing cells were plated into semisolid medium (fresh medium containing 0.3% agar). After 2 weeks plates were analysed for the presence of colonies. Skp 2/H-Ras^{G12V}-expressing cells readily formed colonies in soft agar, which is a strong criterion for cultured cell transformation. In addition, 1×10⁶ Skp 2/H-Ras^{G12V}-expressing cells were injected in the flank of 2-3 week old nude mice. Mice were scored for the presence of tumours at the injection site. At two weeks thereafter, tumour formation was detected in all experimental animals injected with Skp 2/H-Ras^{G12V}-expressing cells but not with control REFs. The results of the cotransfection experiments shows that Skp 2 can act as an oncogene.

### EXAMPLE 2

### Immunohistochemical Analysis of Cells Shows a Significant Inverse Relationship Between the Levels of Skp 2 and p27 in Tumour Cells

Skp 2 expression was analysed in a series of human primary oral squamous cell carcinomas, breast carcinomas, lymphomas and prostate cancers. In general, 5 micrometer thick formalin fixed and paraffin embedded tissue sections were stained for p27 and Skp 2 protein by immunohistochemistry using a monoclonal antibody against p27 and polyclonal antibody against Skp 2.

Monoclonal antibodies against p27 are available from Transduction Laboratories. Polyclonal antibodies against Skp 2 are readily raised by persons of average skill in the art by immunisation of an animal with a suitably purified Skp 2 preparation. The polyclonal antibodies can additionally be affinity purified as described by Lisztwag J et al (1998) EMBOJ 17: 368-363.

The results showed that the expression of p27 and Skp 2 is inversely related in all cancers tested. This confirms that Skp 2 is most likely to function as an oncogene.

These results implicate a substrate-recognition subunit of an SCF ubiquitin protein ligase complex in the development of human cancer.

### EXAMPLE 3

### Isolation and Cloning of a CDNA Encoding an Skp 2 Associated Protein (STAP1)

A yeast-two hybrid screen was performed using Skp 2 as a bait. From this a cDNA was cloned that encodes for a protein of about 18 kDa that we now refer to as STAP1 (for Skp 2-associated protein one). The STAP1 protein is hitherto unknown.

About 1×10⁶ clones were screened from a HeLa cell library constructed in pGAD-GH (Clontech) which baits encoding residues 101-423 of human Skp 2 cloned in the GAL4 DNA-binding domain vector pAS2-1. Interacting clones were identified after selection on triple-dropout media (minus Leu/Trp/His with 25 mM 3-amino-triazole), and assaying for strong-galactosidase activity. 35 positive clones were sequenced. Sequence comparison revealed that all cloned cDNAs encode for the novel protein STAP1, having a molecular weight of about 18 kD.

### EXAMPLE 4

### Production of Recombinant STAP1

Human STAP1 full-length version was expressed in Escherichia coli BL21 as glutathione-S-transferase (GST) fusion proteins and purified on glutathione-sepharose, eluted with glutathione. Methodology is described in Kaelin et al (1991) Cell. 64: 521-532 and also Krek etal(1994) Cell. 78: 161-172.

### EXAMPLE 5

### Preparation of Antibodies Reactive Against STAP1

Eluted STAP1 material from example 4 above was injected into mice to generate monoclonal antibodies. A routine monoclonal antibody production protocol was undertaken as will be well known to those of skill in the art. Polyclonal antiserum and antibodies against STAP1 were also generated by injection of the STAP1 eluted material of example 4 above into rabbits following a standard form of protocol which will be familiar to those of skill in the art.

### EXAMPLE 6

### Immunoprecipitation and Electrophoretic Separation of a Complex Containing STAP1 from HeLa Cells.

Large scale immunoprecipitation was carried out with HeLa whole cell extracts. 100 μg of monoclonal anti-STAP1 antibody coupled to protein A was added to 50 ml of HeLa nuclear extracts (from about 2 to 10⁹) and rotated for 2hr at 4° C. The immunoprecipitates were then washed in 25 ml of TNN [20 mM Tris-HCI (pH 8.0), 0.1 M NaCl, 1 mM EDTA, 0.5% NP-40] four times. The precipitated proteins were eluted with 300 μl 0.2M Glycine (pH 2.5) into Laemmli buffer and separated on a 10% SDS-polyacrylamide gel. The gel was then stained with silver.

### EXAMPLE 7

### Analysis of STAP1-Associated Protein by Mass Spectrometry

The SDS-PAGE separated proteins were excised from the gel of example 6, reduced with DTT, alkylated with iodoacetamide and cleaved with trypsin (Promega, sequencing grade) as described by Shevchenko, A., Wilm, M., Vorm, O. and Mann, M. (1996) Anal. Chem., 68: 850-858. The extracted tryptic peptides were desalted with 5% formic acid, 5% Methanol in H₂0 on a 1 μl Poros P20 column and concentrated to 1 μl with 5% formic acid, 50% Methanol in H₂O directly into the Nanoelectrospray ionisation (NanoESI) needle. NanoESI mass spectrometry (MS) was performed according to the published method of Wilm, M. and Mann, M. (1996) Anal. Chem., 68: 1-8. The mass spectra was acquired on an API 300 mass spectrometer (PE Sciex, Toronto, Ontario, Canada) equipped with a NanoESI source (Protana, Odense, Denmark). See also W. R. Pearson & D. J. Lipman (1998) PNAS, 85: 2444-2448.

The STAP1-containing complex is found to contain a large number, about 20 or so proteins. As well as STAP1, the complex has also been found to comprise TIP48, TIP49 (two evolutionarily conserved ATPases and DNA helicases), RPB5 (RNA plo II subunit 5), RMP1 (RNA plo II mediator protein) and at least three other hitherto unknown proteins.

### EXAMPLE 8

### Analysis of the STAP-containing Complex by Sucrose Density Gradient Centrifugation and Western Blotting

A crude HeLa cell extract was subjected to 5- 30% and 10- 30% (w/v) density centrifugation. The sample was loaded in TNN buffer made up of mM Tris (pH 7.5), 250 mM Na Cl, 0.5% NP40, 1MM DTT, sodium vanadate, PMSF and aproteinin. The buffer was also used in the sucrose gradient but the NP40 was omitted.

Each of the fractions was mixed with sample buffer and subjected to standard Laemlli denaturing SDS-PAGE at 12%. A number of gels were run and then each was blotted with an antibody. Polyclonals against RMP1 and TIP49 were used, as were monoclonals against RPB5, TIP48, STAP1 and Skp 2. The lanes of the blotted gels are aligned with their respective sucrose fractions and what is apparent is that the components of the STAP1-containing complex are clearly associated together and do not form part of the main peak of protein in the gradient. The components of the complex are all found in fractions where higher molecular weight proteins sediment. Skp 2 has a different pattern in the gradient compared to the STAP1-containing complex and this is consistent with Skp 2 being a binding partner for STAPI.

Also noted for the first time is how TIP49 antibodies recognise a doublet on SDS-PAGE. There is an immunologically related TIP49 variant of slightly higher molecular weight.

### EXAMPLE 9

### Screening for Anti-cancer Agents Which are Inhibitors of a STAP1-associated DNA Helicase Complex

Small molecule compounds that disrupt specific interactions between the components of a STAP1-containing TIP49, TIP48, RPB5, RMP1, STAP1 and Skp 2, for example are putative anti-cancer agents. The component proteins of the complex are expressed in Sf9 insect cells using recombinant baculoviruses. All possible combinations of pairwise interactions between subunits of the complex are constructed and used to screen synthetic and natural compounds. In practice, coinfection of insect cells followed by immunoprecipitation with the appropriate antibody provides the complex substrate used in the screening assays. Coimmunoprecipitation between two of the above-noted components indicates a direct interaction and hence a target for disruption of interaction by putative anti-cancer agents. For example, STAP1 and Skp 2 coimmunoprecipiate when coexpressed in this system and provide a binding pair suitable as the basis of a screening assay for synthetic or natural compounds which disrupt that binding in some way. To screen for small molecular compounds, recombinant hexahistidine-tagged STAP1 is purified from insect cells and immobilized to the surface of nickel-coated 96-well plates. Immobilized STAP1 is incubated with purified biotinylated Skp 2 and washed. Subsequently, europium-labelled streptavidin is added. Then, time-resolved fluorescence of europium is monitored in the absence of presence of synthetic chemical libraries and natural products.

### EXAMPLE 10

### Screeninq for Anti-cancer Agents Which are Inhibitors of TIP48 and/or TIP49 ATPase Activity

Recombinant TIP48 and TIP49 are expressed in E. Coli using experimental procedures as described in Makino Y et al (1999) J. Biol. Chem. 274: 15329-15335. Purification of recombinant TIP48 and TIP49, as well as assays for ATPase activity and DNA helicase activity are also as described in Makino Y et al (1999). The purified recombinant proteins are used to screen for natural products or synthetic compounds which interfere with the normal enzymic activities of TIP48 and/or TIP49.

The screening assay is conveniently carried out in microtiter plates. TIP48 and/or TIP49 proteins are placed in the wells and one or both of the enzyme assays are carried out in the presence or absence of compounds from natural or synthetic chemical libraries. Advantageously, an ATPase microassay format can be used as described in Henkel R D et al (1988) Anal. Biochem. 169: 312-318.

### EXAMPLE 11

### Rapamycin Sensitive Signaling on Human RMP1

Human Embryonic kidney (HEK293) cells are grown in DMEM+10% FCS. Exponentially growing HEK 293 cells are deprived of serum overnight, and then induced with 50 nM insulin in the presence or absence of 20 nM rapamycin. Protein extracts were prepared from cells harvested at 0 h, 1 h, 2 h, 4 h and 8 h after insulin induction. The cell extracts were subjected to SDS-polyacrylamide gel electrophoresis and the resulting gels were analyzed by immuno-blotting using a rabbit polyclonal anti-human RMP antibody. Antibodies specific for RMP are easily prepared using standard laboratory practices. A secondary antibody specific for rabbit antibodies, labelled was used to visualize RMP on the immunoblot using routine procedures.

A slower migrating form of RMP is induced by insulin after 1 h induction, which is maintained throughout the 8 h time course of the experiment, with a loss/disappearance of the fast migrating form and gradual loss of the slow migrating form In the presence of 20 nM rapamycin formation of this slower migrating form is completely blocked, with the fast-migrating form being maintained over the time course of the experiment.

These findings suggest RMP1 as a novel substrate of the insulin signalling pathway and the TOR/S6 kinase pathway. The rapamycin sensitivity of the RMP signalling suggests that TOR or S6 kinase might be good candidate kinases controling the formation of the novel insulin induced form of RMP1. In the presence of insulin and amino acids (in the culture medium in this example), RMP is present as a slower migrating form, indicating a phosphorylated form. Under these conditions, the cells continue to synthesize amino acids. With the addition of rapamycin, RMP is present as a fast migrating form and blocks the synthesis of amino acids

All references referred to herein, as well as priority application GB 0011439.7 filed May 12, 2000, are hereby incorporated by reference as if each were referred to individually.

1. A protein or peptide comprising at least 5 consecutive amino acids of SEQ ID NO: 3.

2. The protein or peptide of claim 1, comprising at least 10 consecutive amino acids of SEQ ID NO: 3.

3. The protein or peptide of claim 1, comprising at least 15 consecutive amino acids of SEQ ID NO: 3.

4. The protein or peptide of claim 1, comprising at least 20 consecutive amino acids of SEQ ID NO: 3.

5. The protein or peptide of claim 1, comprising the sequence as set forth in SEQ ID NO: 4.

6. The protein or peptide of any preceding claim, wherein said protein or peptide is phosphorylated.

7. An antibody that specifically recognizes the protein or peptide of any of claims 1 to 5.

8. An antibody that specifically recognizes the phosphopeptide or phosphorylated protein of claim 6.

9. The use of an antibody of claim 8 for the manufacture of a medicament for the prophylaxis or treatment of cancer.

10. An antibody of claim 8 for use as pharmaceutical.

11. A nucleic acid encoding the protein or peptide of any of claims 1 to 5 or its complement.

12. The nucleic acid of claim 11, wherein said nucleic acid comprises at least a fragment of the sequence as set forth in SEQ ID NO: 5, or its complement.

13. The nucleic acid of claim 11, wherein said nucleic acid comprises the sequence as set forth in SEQ ID NO: 6 or its complement.

14. A nucleic acid antisense to a part of a nucleic acid of SEQ ID NO: 6, or antisense to a sequence having at least 70% homology and capable of hybridizing with SEQ ID NO: 6 under low stringency conditions.

15. An antisense nucleic acid as claimed in claim 12, wherein the sequence to which the nucleic acid is antisense to has at least 80% homology with SEQ ID NO: 6, preferably at least 90%, more preferably at least 95%, even more preferably at least 95%, most preferably at least 99%.

16. An antisense nucleic acid as claimed in claim 12 or claim 13, wherein at least some of the nucleotide residues are resistant to nuclease degradation and selected from phophorothioates and/or methylphosphonates, for example.

17. An antisense nucleic acid as claimed in any of claims 12 to 14 for use as a pharmaceutical.

18. The use of an antisense nucleic acid of any of claims 12 to 14 for the manufacture of a medicament for the prophylaxis or treatment of cancer.


SEQUENCE LISTING (7 sequences)

SEQ ID NO: 1 - protein, 157 aa, Homo sapiens
MATPPKRRAVEATGEKVLRYETFISDVLQRDLRKVLDHRDKVYEQLAKYLQLRNVIERLQEAKHSELYMQVDLGCNFFVDTVVPDTSRIYVALGYGFFLELTLAEALKFIDRKSSLLTELSNSLTKDSMNIKAHIHMLLEGLRELQGLQNFPEKPHH

SEQ ID NO: 2 - DNA, 726 bp, Homo sapiens
  CDS (156)..(665)
ggaggtaaaggccgcgcttgggtgtccctgggtggtcgggtccccgagttgggaggggcggaaggctgaacctccagcttgagccggacaagccgattcccagcgttgagagggtagagatgaactgtgtgtgaggccaaactggatcggtcaacatggtcttccccctccccactccccaggagcccatcatggcgacgccccctaagcggcgggcggtggaggccacgggggagaaagtgctgcgctacgagaccttcatcagtgacgtgctgcagcgggacttgcgaaaggtgctggaccatcgagacaaggtatatgagcagctggccaaataccttcaactgagaaatgtcattgagcgactccaggaagctaagcactcggagttatatatgcaggtggatttgggctgtaacttcttcgttgacacagtggtcccagatacttcacgcatctatgtggccctgggatatggttttttcctggagttgacactggcagaagctctcaagttcattgatcgtaagagctctctcctcacagagctcagcaacagcctcaccaaggactccatgaatatcaaagcccatatccacatgttgctagaggggcttagagaactacaaggcctgcagaatttcccagagaagcctcaccattgacttcttccccccatcctcagacattaaagagcctgaaaaaaaaaaaaaaaaaaaaaaaaaa

SEQ ID NO: 3 - protein, 25 aa, Homo sapiens
MEAPTVETPPDPSPPSAPAPALVPL

SEQ ID NO: 4 - protein, 534 aa, Homo sapiens
MEAPTVETPPDPSPPSAPAPALVPLRAPDVARLREEQEKVVTNCQERIQHWKKVDNDYNALRERLSTLPDKLSYNIMVPFGPFAFMPGKLVHTNEVTVLLGDNWFAKCSAKQAVGLVEHRKEHVRKTIDDLKKVMKNFESRVEFTEDLQKMSDAAGDIVDIREEIKCDFEFKAKHRIAHKPHSKPKTSDIFEADIANDVKSKDLLADKELWARLEELERQEELLGELDSKPDTVIANGEDTTSSEEEKEDRNTNVNAMHQVTDSHTPCHKDVASSEPFSGQVNSQLNCSVNGSSSYHSDDDDDDDDDDDDNIDDDDGDNDHEALGVGDNSIPTIYFSHTVEPKRVRINTGKNTTLKFSEKKEEAKRKRKNSTGSGHSAQELPTIRTPADIYRAFVDVVNGEYVPRKSILKSRSRENSVCSDTSESSAAEFDDGRGVLRSISCEEATCSDTSESILEEEPQENQKKLLPLSVTPEAFSGTVIEKEFVSPSLTPPPAIAHPALPTIPERKEVLLEASEETGKRVSKFKAARLQQKD

SEQ ID NO: 5 - DNA, 75 bp, Homo sapiens
atgcacgtgtgagatgcggcagcgggcggcgcggacgcgaacagcagcggcggcggcgggcgcggcctcctgggc

SEQ ID NO: 6 - DNA, 2519 bp, Homo sapiens
  CDS (280)..(1881)
ctggctgggcccgcaccggagaggcgtctcggtacctggcaggcggcctgctactcggagcccgctgcggggcggcggcggcggggacatgcacgtgtgagatgcggcagcgggcggcgcggacgcgaacagcagcggcggcggcgggcgcggcctcctgggcgcggggcgcgcggtgcctgagggcgggcgcgcgggcgctgggcaactgccggccgcgccgcctgcgcaggcgctggttcaggactcacacgccgcgctgaggcccgcgggcccgtcatggaggcgcccaccgtggagacgccccccgacccctcgcccccttcggccccggcccctgccctggttccgttgcgcgccccggatgtggcgcggctgcgcgaggagcaggaaaaggtggtcactaactgccaagagagaatccagcattggaagaaggtagataatgactataatgcccttcgagaaagactcagcaccttgcctgataaattgtcttataatataatggtaccatttggcccttttgccttcatgccaggaaaacttgtccatactaatgaagtcactgttttactgggggacaactggtttgcaaagtgctcagcaaagcaggctgtaggtttagttgagcaccggaaagaacatgtaagaaaaacaatagatgacttaaaaaaagtgatgaaaaattttgaatccagagttgaattcacagaagatttgcagaaaatgagcgatgctgcaggtgatattgttgacatacgagaagaaattaaatgtgacttcgaatttaaagcaaaacaccgaattgctcataaaccgcattccaaaccaaaaacttcagatatttttgaagcagatattgcaaatgatgtgaaatccaaggatttgctagctgataaagaactgtgggctcgacttgaagaactagagagacaggaagaattgctgggtgaacttgatagtaagcctgatactgtgattgcaaatggagaagatacgacatcttctgaagaggaaaaggaagatcgtaacacaaatgtgaatgcgatgcatcaagtaacagactctcatactccttgtcataaggatgttgcaagttcagaaccattcagtggtcaagtgaatagtcagttgaactgttcagtgaatggttccagttcttaccacagtgatgatgatgatgatgatgatgacgacgacgacgacaacattgacgacgatgatggtgataacgaccatgaggctttaggggttggagataattctataccaacaatatatttttcacatactgttgagcctaagagggtccgaataaatactggaaagaataccactttaaaattcagtgaaaagaaagaagaagccaaacgtaaacgaaagaacagcactggcagtggccactctgcccaggagctgccgaccatcaggacgcctgcagacatttacagagcctttgttgatgttgtgaatggagaatatgtccctcgcaaatccatcttgaagtctcgaagtagagagaatagtgtgtgtagcgacactagtgaaagcagtgctgctgaatttgatgatgggcggggagttttgaggagtatcagctgcgaagaagccacttgcagtgacaccagtgagagcattttggaagaggaaccacaagaaaatcaaaagaaacttttgcccttatcagtaacacctgaggctttttctggaactgttatagaaaaagaatttgtatcaccttccttaacaccacccccagccattgctcatcccgcactacccactattccagaacgaaaggaagttctgttggaagcatctgaagaaactggaaagagggtttcaaagtttaaagctgccagattgcaacagaaagactaggccctgtctaggaaatgggaatttacatcctaaaacctagttgttcatttgtttagagtatctatagcaaaataggttacatgtagtttgaaataaggtatcctgagttactttggcaacaagttcttttacccttacccgtggtatttgaaaaaaatcaaggtaactgtctgaatactttaatatcagcttgttttgtgaattctctgaatactgtcaacactcttatctaagtttgcctttatgatgcagtggcagcattttgaattacttttcaaagaatactgttcatatgcattgtttttgtgtttcaaactaaatacaggcagttttgtgccagctgtgatattgtgcataccatatggaccattttaaagaaaattttaaaatttcaaatagattcaacaattacattacttgctttcacattttaaaggcactttaaaaaaatctacttctcttgtaggttttgcggctagttggctattcaagaaacctcgcccctctgaatgtcatactgtaatctttaaggaagaaagctacatgaattaattgtactctatgggaaaatttctttggaaagatattttgtaaaacttttttttccaagtaaaaactttatgaaacttggtctc

SEQ ID NO: 7 - protein, 169 aa, Homo sapiens
MVFPLPTPQEPIMATPPKRRAVEATGEKVLRYETFISDVLQRDLRKVLDHRDKVYEQLAKYLQLRNVIERLQEAKHSELYMQVDLGCNFFVDTVVPDTSRIYVALGYGFFLELTLAEALKFIDRKSSLLTELSNSLTKDSMNIKAHIHMLLEGLRELQGLQNFPEKPHH